(19) United States
(12) Patent Application Publication
(10) Pub. No.: US 2014/0255502 A1
(43) Pub. Date: Sep. 11, 2014

(54) NANOPARTICLE DRUG CARRIER, PHARMACEUTICAL COMPOSITION AND MANUFACTURING METHOD THEREOF

(71) Applicant: National Chiao Tung University, Hsinchu City (TW)

(72) Inventors: San-Yuan Chen, Hsinchu City (TW); Wei-Ming Li, Kaohsiung City (TW)

(73) Assignee: National Chiao Tung University, Hsinchu City (TW)

(21) Appl. No.: 14/036,703

(22) Filed: Sep. 25, 2013

(30) Foreign Application Priority Data
Mar. 6, 2013 (TW) 102107954

(51) Int. Cl. A61K 9/51; A61K 9/50

(52) U.S. Cl. CPC A61K 9/51; A61K 9/5094
USPC 424/492; 514/34; 514/449; 514/283; 514/679

(57) ABSTRACT

A pharmaceutical composition is provided. The pharmaceutical composition includes a nanoparticle, a shell and a drug, wherein the nanoparticle has an outer surface and the drug is mixed with the shell and is formed on the outer surface.

### CROSS REFERENCE TO RELATED APPLICATIONS

This application claims benefit under 35 U.S.C. §119 of TW Application No. 102107954, filed Mar. 6, 2013, the contents of which are incorporated by reference as if fully set forth.

### FIELD OF THE INVENTION

The present invention relates to a nanopartical drug carrier, especially relates to a drug carrier including an amphiphilic nanoparticle and a calcium phosphate shell for controlling the release of the drug.

### BACKGROUND OF THE INVENTION

In recent years, nano-materials and nanotechnology have become the key point for research in every nation. A nanomaterial usually refers to a material with a size range from 1 nm to 100 nm. Typically, recent developments in biomedicine are combined with nanoscience to bring both medical therapy and gene therapy from the ex vivo experiment in the lab to the cell level or molecular biology level, and then to the living body (in vivo). Considering the usage safety in the living body, a nanoparticle used to coat a drug must meet criteria such as non-biotoxicity, water solubility and biocompatibility.

Calcium phosphate (CaP) has long been well recognized as a highly biocompatible bioactive substance because it is a critical precursor for bone growth, and thus can be used in the biomedical field. CaP is able to bind and encapsulate drugs or nucleic acids, and it protects the encapsulated drug from enzymatic degradation during delivery into cells. Because of these advantages, CaP is used as the shell of the drug carrier to enable the drug encapsulated in the core to be delivered to the target cells.

Yurong C. et al use surfactant micelle to encapsulate a drug, deposit CaP on the surface of the micelle to form a nano-shell, and make the shell collapse by ultrasound to release the drug (Chem Mat. 2007; 19(13) 3081-3083). Rim H P et al. carry drug molecules by mesoporous silica nanocontainers and cover the surface of the silica nanospheres with CaP as the shell to form the drug carrier. The existence of the CaP shell is to prevent the drug from leaking from the pores of the silica nanospheres and to provide a controlled releasing effect. (Angewandte Chemie-International Edition. 2011; 50:8853-8857).

There are some important problems in the application of CaP as a nanoparticle drug carrier. For example, (i) the drug needs to be absorbed by the core of the drug carrier (i.e. the nanoparticle) first and then the CaP covering is subsequently performed, and the drug tends to leak from the drug carrier during this procedure such that the encapsulation efficiency (EE) is low; (ii) in the current developments regarding drug carriers, the CaP is quickly deposited on the surface of the drug carrier, such that it is hard to add iron oxide nanoparticles or functonalized nanoparticles thereon, and thus the carrier usage is limited; and (iii) the desired goals of controlled drug release and releasing amount manipulation cannot be achieved although the drug molecule in the nanoparticle can be released using another reaction (such as natural diffusion). Therefore, it is desired to develop a functionalized drug carrier that can control the drug release and monitor the position of the drug carrier synchronically.

In view of the drawbacks of the prior art, the present invention develops a pharmaceutical composition which has an iron oxide@amphiphilic gelatin nanosphere as the core and pH-sensitive CaP as the shell covering on the surface of the core, to produce an iron oxide/amphiphilic gelatin@CaP core-shell nanostructure. A drug molecule is added simultaneously with the CaP formation and is perfectly encapsulated in the layer of the shell, to complete the pharmaceutical composition. In addition, a hydrophobic drug could be easy encapsulated in the core during the process of emulsion to form iron oxide/hydrophobic drug/amphiphilic gelatin nanosphere, and then a hydrophilic drug molecule is added simultaneously with the CaP formation. Finally the iron oxide/hydrophobic drug/amphiphilic gelatin@CaP core/shell nanostructure is capable of enwrapping both hydrophobic and hydrophilic drug and control the drug release in different release model such as sequential release of the hydrophobic and hydrophilic drugs in a various acidic environment around the tumor cells. The summary of the present invention is below.

### SUMMARY OF THE INVENTION

In the drug carrier described in the present invention, a core and a CaP shell are included, wherein the core includes a magnetic nanoparticles and an amphiphilic gelatin that coats the magnetic nanoparticles, and the CaP shell is configured outside the core and used to store a water soluble drug.

In another aspect, the present invention describes a pharmaceutical composition including a nanoparticle having an outer surface, a shell and a drug, wherein the drug is mixed with the shell to be formed on the outer surface of the nanoparticle.

In addition, the present invention also describes a method for preparing a pharmaceutical composition, including providing a nanoparticle having an outer surface, providing a shell material, providing a drug to be mixed with the shell material to form a mixture, and enabling the mixture to be attached to the outer surface.

Using the property of the material and the combination of the drug, the present invention designs and prepares a multi-functional drug carrier that has the effect of controlled release and monitors the position of the drug carrier synchronically. The drug carrier maintains good structural stability in the neutral or basic solution, and controls the local release of the drug by dissolving the CaP shell of the drug carrier under the acidic environment. Thereby, for example, a large amount of an anti-cancer drug can be accumulated at the site of a tumor to achieve the effect of tumor treatment. Meanwhile, the preparation method described in the present invention modifies the traditional method that requires two stages, drug loading and shell formation, as simultaneously completed in one step, which is advantageous over the prior arts.

The hydrophobic drug could be easily encapsulated in the core during the process of emulsion to form the dual drugs carrier. The use of core/shell nanoparticles combined two therapeutic drugs could enhance the effective therapy in the tumors.

Other objects, advantages and efficacies of the present invention are described in detail below and taken from the preferred embodiments with reference to the accompanying drawings.

### BRIEF DESCRIPTION OF THE DRAWINGS

FIG. 1 is a flow chart for preparing the drug carrier of the present invention.

FIG. 2 is a Fourier transform infrared (FT-IR) spectra of the drug carrier of the present invention.

FIG. 3 is a diagram showing the superconducting quantum interference device (SQUID) measuring results of the drug carrier of the present invention.

FIG. 4 is a diagram showing the Magnetic resonance imaging (MRI) results of the drug carrier of the present invention.

FIG. 5(A) is a flow chart for preparing the drug carrier of the present invention that encapsulates one drug.

FIG. 5(B) is a flow chart for preparing the drug carrier of the present invention that encapsulates two drugs.

FIG. 6 is a diagram showing the drug release profiles under different acidic levels after the drug carrier of the present invention encapsulates the drug.

FIG. 7 is a diagram showing the toxicities of the amphiphilic gelating/iron oxide/CaP core-shell nanocarrier of the present invention and iron oxide@amphiphilic gelatin nanosphere in HeLa cells.

FIG. 8(A) shows the hydrophobic drug (Taxol) release under different acidic levels when the drug carrier of the present invention encapsulates two drugs.

FIG. 8(B) shows the hydrophilic drug (DOX) release under different acidic levels when the drug carrier of the present invention encapsulates two drugs.

FIG. 9 shows the drug release profiles of Curcumin (CUR) and Doxorubicin (DOX) from drug carriers of the present invention under different acidic levels.

### DETAILED DESCRIPTION OF THE PREFERRED EMBODIMENTS

Further embodiments herein may be formed by supplementing an embodiment with one or more elements from any one or more of the other embodiments herein, and/or substituting one or more elements from one embodiment with one or more element from one or more of the other embodiments herein.

### EXAMPLES

The following non-limiting examples are provided to describe particular embodiments. The embodiments throughout may be supplemented with one or more details from one or more examples below, and/or one or more elements from an embodiment may be substituted with one or more details from one or more of the examples below.

The present invention provides a drug carrier having a basic core-shell structure, wherein the core includes a magnetic material and an amphiphilic gelatin that coats the magnetic nanoparticles, and the shell is made of calcium phosphate and configured outside the core to store a water soluble drug. The magnetic nanoparticles in the drug carrier concurrently have the effects of magnetic guidance and magnetic imaging, which enables the drug carrier of the present invention to be applied in tracking tumors or other tissues. In the embodiments of the present invention, the magnetic nanoparticle is preferably Fe₃O₄ because iron oxide has outstanding magnetic sensitivity and a superparamagnetic property. The amphiphilic gelatin used in the present invention is modified with hexanol anhydride, such that the hydrophobic magnetic nanoparticle is encapsulated in the hydrophobic layer inside the amphiphilic gelatin during the procedure of encapsulating the magnetic material. As a result, the carboxyl groups of the amphiphilic gelatin expose and enable a negative surface charge and good dispersion of the spheroid. In the present invention, the amphiphilic gelatin polymer has a molecular weight ranging from 87,000 to 100,000 g/mol and dissolves in various solvents at different concentrations (0.01 μg/ml to 1000 mg/ml), wherein the various solvents may be a water solution, an organic solvent or any mixing solution. The size of the iron oxide/amphiphilic gelatin spheroid of the present invention ranges from 50 nm to 300 nm, and the size of the iron oxide nanoparticle ranges from 5 nm to 20 nm.

The core of the drug carrier of the present invention is an amphiphilic spheroid, which provides attracting force for the combination of the CaP shell so as to attach the CaP outside the core. After the drug carrier is formed, its size ranges from 100 nm to 400 nm and the thickness of the shell ranges from 5 nm to 100 nm. Therefore, the drug carrier of the present invention can be applied in the living body, such as mammals, to control the local release of the drug in the body and thus achieve the effect of treating tumors locally.

The differences between the present drug carrier and the prior arts reside in that the amphiphilic nanoparticle is coated with the CaP shell, and the drug is stored in the shell simultaneously. Such a way of forming the drug carrier may prevent drug leakage while being compared with the traditional technique that encapsulates the drug in the core. In addition, the drug carrier of the present invention has good pH-sensitivity that can release large amounts of the drug quickly and precisely by using the differences in the environmental pH values. When the external environment is neutral or basic, the drug carrier can encapsulate the drug in the shell layer continually to prevent the drug from natural release, and this outstanding property is of great benefit to the control of the drug release. However, when the external environment becomes acidic, the drug molecule is released due to the breakdown of the CaP, and a higher acidic level enables the faster breakdown rate of the CaP, and thus the drug release rate increases. It can be seen that the CaP shell in the drug carrier of the present invention is not only used to store the drug but controls the release rate for the drug in accordance with the pH value.

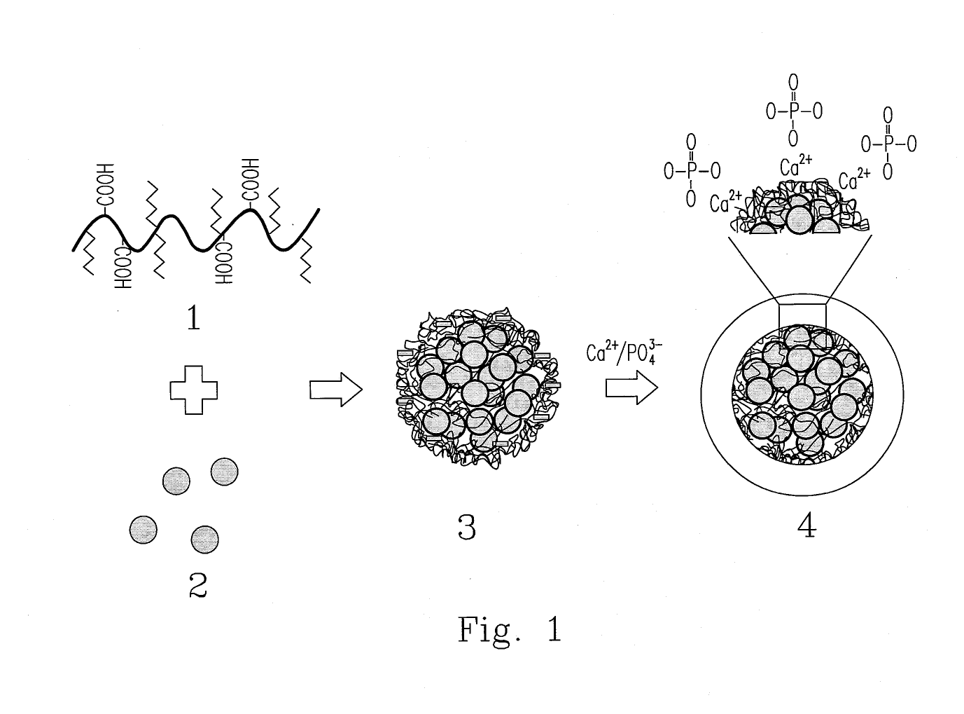

FIG. 1 is a flow chart showing the preparation of the drug carrier of the present invention. As shown in FIG. 1, the gelain, which is a protein polymer, is modified with a hydrophobic functional group (i.e. a hexanoyl group in the present invention) as an amphiphilic gelain 1 having the hydrophilic/hydrophobic dual property. The amphiphilic gelain 1 is mixed with the iron oxide nanoparticle 2 to form an iron oxide/amphiphilic gelatin nanosphere 3, to encapsulate the iron oxide nanoparticle in the hydrophobic layer inside the amphiphilic gelatin and expose the carboxylate groups. The iron oxide/amphiphilic gelatin nanosphere is mixed with an aqueous solution containing calcium and phosphate ions to form a CaP shell on the outer surface of the iron oxide/amphiphilic gelatin nanosphere, which becomes the drug carrier 4 of the present invention, the iron oxide/amphiphilic gelatin@CaP core-shell structure.

The embodiments for preparing the drug carrier of the present invention are described in detail below.

Preparation of Iron Oxide/Amphiphilic Gelatin Nanosphere (AGIO)

1.5 g of gelatin (type A) is taken in a 100 ml flask, and 20 ml of pure water and 1 ml of 0.1N NaOH are added thereto to be stirred for 4 hours at 60° C. so as to dissolve completely. After 20 ml of ethanol is added and mixed evenly, various concentrations of hexanoic anhydride are added to the mixture and the mixture is stirred for 5 hours at 60° C. to obtain the hydrophilic/hydrophobic amphiphilic gelatin polymers with different grafting ratios (see Table 1), wherein the grafting ratio ranges from 0% to 100%.

TABLE 1

| | Hexanoic | Critical aggregation | Zeta | | |
|---|---|---|---|---|---|
| | anhydride | concentration(CAC) | potential | Conversion | Mean size |
| Sample | (M) | (×10⁻³ mg/ml) | (mV) | (%) | (nm) |
| GM1 | 0.2 | 10 ± 0.38 | −9.3 ± 1.3 | 49.6 ± 4.2 | 58.4 ± 8.8 |
| GM2 | 0.4 | 7.08 ± 0.36 | −15.5 ± 1.8 | 68.6 ± 5.5 | 80.2 ± 8.2 |
| GM3 | 0.6 | 3.16 ± 0.42 | −24.4 ± 2.0 | 85.8 ± 4.7 | 175.3 ± 7.4 |
| GM4 | 0.8 | 2.16 ± 0.35 | −28.2 ± 2.5 | 97.7 ± 2.2 | 285.2 ± 12.8 |

0.1 mg of amphiphilic gelatin is dissolved in 2 ml of aqueous solution, the iron oxide nanoparticles in an oil phase are dissolved in 0.5 ml of chloroform (CHCl₃), and the iron oxide solution is evenly mixed with the 2 ml solution of the amphiphilic gelatin molecules. The mixed solution is emulsified using an ultrasonicator and will slowly turn light brown. After being sonicated for 1 minute, the solution is placed on a hot plate to be heated to 60° C. After the organic phase solvent (CHCl₃) has completely evaporated, the mixed solution changes from light brown to black, and the precipitate is collected in a centrifuge and washed with deionized water several times. Finally, the precipitate is dissolved in the deionized water for storage, and thus the AGIO structure is obtained.

It can be observed by a transmission electron microscope that the iron oxide nanoparticle is indeed encapsulated by the amphiphilic gelatin to form the iron oxide nanospheres. It can be seen that the particle size of the iron oxide nanoparticle is about 4.8 nm, and the particle size of the AGIO is about 80 nm. In addition, it can be seen that the particle size distribution of the amphiphilic gelatin/iron oxide nanosphere measured by dynamic light scattering (DLS) is very centralized, which means that the sizes of the nanoparticles prepared by this method are uniform. As shown in Table 1, various sizes of the AGIO particles can be generated by encapsulating the iron oxide nanoparticles in the oil phase with the amphiphilic gelatin molecules having different grafting ratios.

Preparation of iron oxide/amphiphilic gelatin@CaP core-shell nanostructure (AGIO@CaP)

5 ml of the deionized water is mixed with 60 μL of 0.1M (NH₄)H₂PO₄ and 100 μL of 0.1 M NaOH (pH 10). 2 mg of amphiphilic gelatin/iron oxide nanospheres mixed with 100 μL of 0.1 M CaCl₂ is dropwise added to the above phosphate-containing solution. After the mixture is stirred for 30 minutes, the white precipitate is collected in a centrifuge and washed with a buffer solution (pH 7.4) several times. The collected precipitate is dissolved in the buffer solution (pH 7.4) to obtain the AGIO@CaP structure.

According to the embodiments in the present invention, the calcium and phosphate ions for preparing the CaP shell are provided by CaCl₂ and (NH₄)H₂PO₄. However, the skilled person in the art can replace these solutions with other solutions that can provide calcium and phosphate ions.

A TEM image of the drug carrier of the present invention shows that the CaP shell successfully coats the surface of the AGIO and the drug carrier has good dispersibility and a size of about 120 nm. When analyzing the structure of the CaP by a high-resolution TEM, no lattice is observed, and thus the synthesized CaP has an amorphous structure.

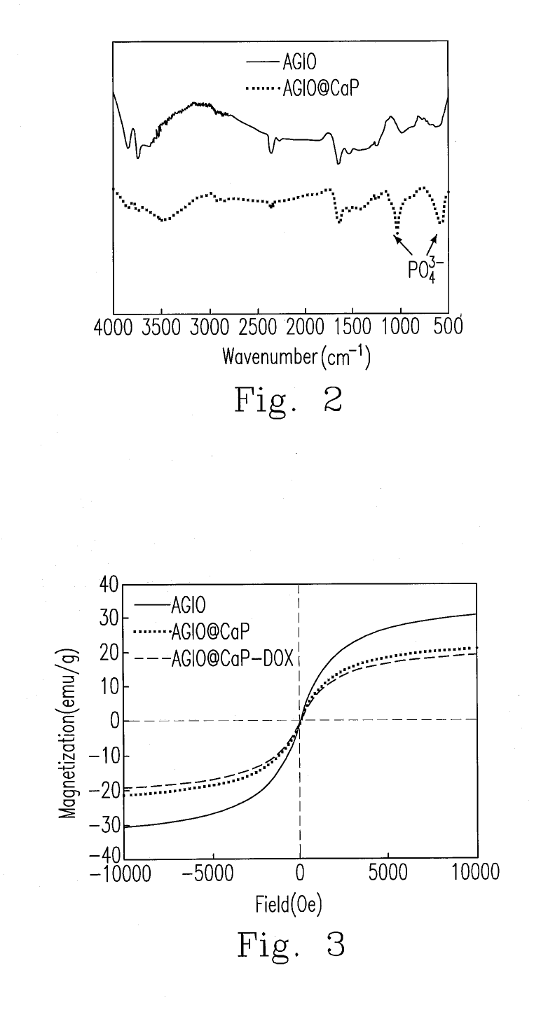

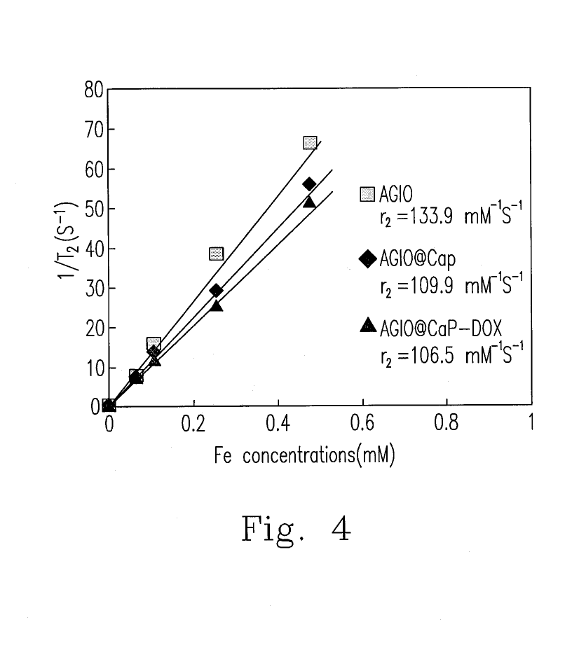

Regarding the structural identification, Fourier transform infrared (FT-IR) and energy dispersive X-ray (EDX) spectroscopy were used to analyze the structure and components of the CaP. As shown in FIG. 2, the absorption band at 680 cm⁻¹ is the characteristic peak of the iron oxide, and the absorption bands at 608 and 1043 cm⁻¹ are the characteristic peaks of the phosphate inside the CaP. The Ca/P molar ratio of 1.5 obtained by EDX spectroscopy suggests that the shell is an amorphous calcium phosphate (ACP). As shown in FIG. 3, the magnetic sensitivity characteristics of the drug carrier of the present invention was measured using a superconducting quantum interference device (SQUID), and the iron oxide/amphiphilic gelatin@CaP drug carrier has excellent superparamagnetic and magnetic sensitivity characteristics, even though the carrier encapsulates a drug (AGIO@CaP-DOX). FIG. 4 is a diagram showing the Magnetic resonance imaging (MRI) results of the drug carrier of the present invention. As shown in FIG. 4, the transverse relaxivities of the drug carrier of the present invention is about 109 s⁻¹mM⁻¹ Fe, proving the MR imaging characteristics.

According to the present invention, the drug can be mixed into the CaP shell in the process of forming the drug carrier, so as to be commonly formed on the outer surface of the nanoparticle and become a pharmaceutical composition. Since the outer surface of the amphiphilic nanoparticle of the present invention is hydrophilic and carries a negative charge, the calcium ions are adsorbed around the outer surface of the amphiphilic nanoparticle due to electrostatic interactions, and the phosphate ions subsequently deposit thereon. The water soluble drug carrying the positive charge is mixed with the CaP to be commonly attached to the outer surface of the amphiphilic nanoparticle, and thus a CaP shell storing the drug therein is formed.

The way to encapsulate the drug in the present invention is co-deposition in that the drug molecule is added at the same time that the CaP is formed and encapsulating the drug molecule in the shell in the process of forming the CaP shell. The AGIO@CaP drug carrier developed in the present invention has good pH sensitivity characteristics and can quickly and precisely release the drug using the differences in the environmental pH values. When the external environment is neutral or basic, the drug carrier can encapsulate the drug in the shell layer continually, and this outstanding property is of great benefit to the drug control for a long time. Usually, the pH value of the human body is neutral, but the environment surrounding the tumor is acidic. Since the CaP has a pH-sensitive property, the differences in pH values of the external environment will influence the dissolving rate of the CaP shell layer. The more acidic the environment is, the faster the CaP dissolves, and thus the release rate of the drug can be controlled.

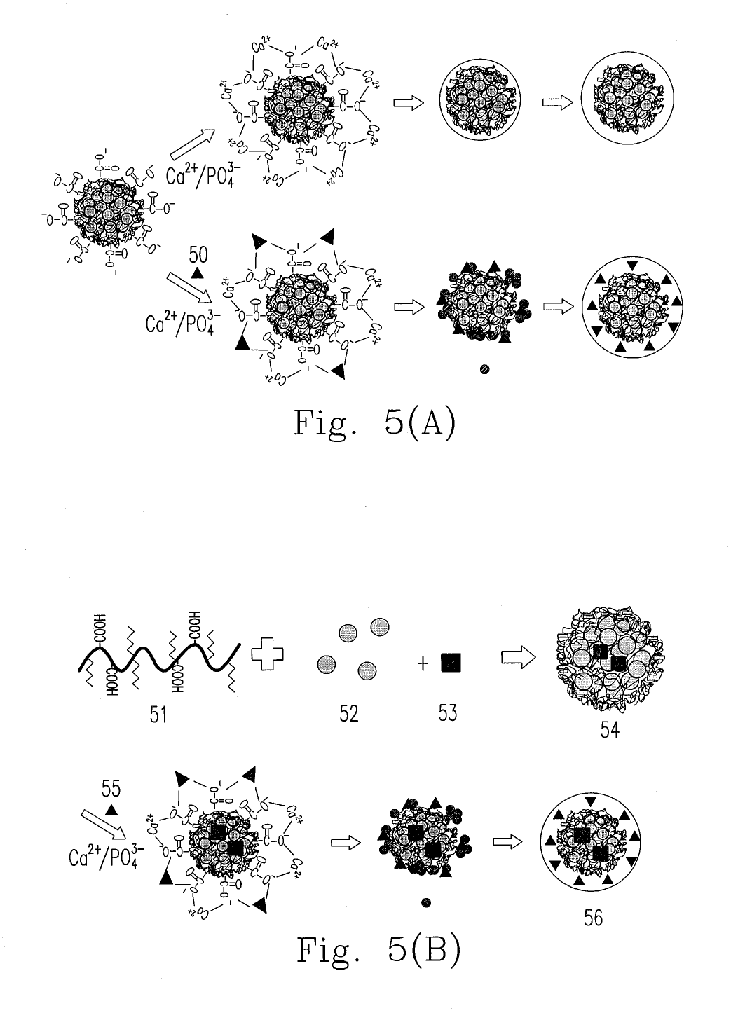

Please refer to FIGS. 5(A) and 5(B) showing two schemes provided in the present invention for encapsulating the drug. FIG. 5(A) shows the first scheme of the present invention for encapsulating the drug. First, the preparation of AGIO is completed, and the CaP being the material of the shell is deposited on the outer surface of the AGIO if the drug is not added thereinto, to become the AGIO@CaP. If a drug 50 is added while forming the CaP shell, the drug 50 is mixed with the CaP to form a mixture and they are commonly attached to the outer surface of the AGIO. In the present invention, the electric charge on the outer surface of the AGIO is opposite to that of the mixture of the drug 50 and the CaP, and thus the two parts combine with each other by electrostatic force. According to the embodiments of the present invention, the drug is preferably a water soluble drug, including at least one of Doxorubicin and its derivatives.

According to an embodiment of the present invention, the AGIO is negatively charged on its outer surface. As shown in FIG. 5(A), free Ca²⁺ cations adsorb around the core by electrostatic interactions between the carboxyl group and calcium ions, which causes CaP to form a thin layer outside the core. As the reaction proceeds, a thicker shell is formed. When the drug and CaP are co-deposited on the surface of the core at the same time, the shell mixed by the CaP and the drug is formed because both positively charged drug and Ca²⁺ simultaneously react with the negatively charged carboxyl groups in the core.

FIG. 5(B) shows the second scheme of the present invention for encapsulating the drug. When the AGIO 54 is formed, the amphiphilic gelatin 51 and the iron oxide nanoparticle 52 are mixed with a first drug 53, to encapsulate the first drug 53 in the AGIO 54. Then, the CaP shell is formed according to the process of the present invention as described above, and a second drug 55 is added thereto simultaneously, causing the second drug 55 to be mixed with the CaP, and they are commonly attached to the outer surface of the AGIO 54 to form the pharmaceutical composition 56. In the present invention, the first drug 53 has a different property from the second drug 55. For example, the first drug 53 is a hydrophobic drug and the second drug 55 is a hydrophilic drug. According to the embodiments in the present invention, the first drug is at least one selected from a group consisting of Taxol, Taxol derivative, Camptothecin, Camptothecin derivative, Curcumin and Curcumin derivative.

According to the embodiment depicted in FIGS. 5(A) and 5(B), the core of the drug carrier of the present invention includes an amphiphilic gelatin and an iron oxide nanoparticle, wherein the existence of the iron oxide nanoparticle enables the drug carrier to have the effects of magnetic guidance and magnetic imaging. However, it should be realized by the skilled person that the iron oxide nanoparticle may be an other suitable hydrophobic molecule. Moreover, the method of encapsulating the drug in the present invention is also adapted to a situation that does not contain the iron oxide nanoparticle, i.e. a situation where a hydrophbic drug is encapsulated in the amphiphilic gelatin and a water soluble drug is mixed with the shell.

The various specific drugs (Doxorubicin, Taxol, Camptothecin, Curcumin etc.) and solutions used in the above preparations are only exemplary embodiments which do not limit the scope of the present invention. For example, the skilled person in the art should realize that Doxorubicin used in the above embodiments can also be its derivatives. In addition, the term “drug” used herein includes but is not limited to clinical drugs, and other active molecules can also be used as the substances to be released in the drug carrier.

The encapsulating test for the anticancer drug, Doxorubicin (DOX)

1 mg of drug molecules, 2 mg of AGIO and 100 μL of 0.1M CaCl₂ are mixed, and the mixture is dropwise added to the above phosphate-containing solution. After the mixture is stirred for 1 hour, the drug molecule is embedded in the shell layer formed by the CaP. Finally, the product is washed with a buffer solution (pH 7.4) several times to obtain the pharmaceutical composition of the present invention.

A TEM image of the drug carrier of the present invention after encapsulating the drug, shows that the CaP shell successfully encapsulates on the surface of the AGIO and the drug carrier has a good dispersibility and a size of about 160 nm. In addition, it is found that the presence of the drug molecule affects the structure of the deposited CaP. The CaP deposits on the surface of the AGIO completely and successively to form a smooth thin shell, if there is no drug molecule. However, the CaP and the drug molecule co-deposit and the drug molecule is encapsulated in the shell at the same time when a drug molecule is added. Therefore, in the early stage of the reaction, small aggregates are first formed around the AGIO and the thickness of the shell grows with increasing reaction time.

In addition, the addition of different doses of CaCl₂ and (NH₄)H₂PO₄ enable the CaP shell of the drug carrier to have different thicknesses, and thus the encapsulation efficiency (EE %) of the drug carrier also increases with the thickened shell, as shown in Table 2. When the Phosphate precursor was change for different ratio, the Ca/P ratio in the composition ranges from 1.1 to 1.6 as shown in Table 3. While preparing a pH sensitive drug carrier of the present invention, the manufacturing technique for the nanomaterial is utilized to control the structure of the drug carrier so as to display the best characteristics.

TABLE 2

| | | | AGIO@CaP | | Encapsulation |
|---|---|---|---|---|---|
| sam- | CaCl₂ | (NH₄)H₂PO₄ | Mean size | CaP Shell | Efficiency |
| ple | (0.1M) | (0.1M) | (nm) | size (nm) | (EE %) |
| 1 | 50 | 30 | 100 ± 10 | 10 ± 5 | 19 |
| 2 | 100 | 60 | 160 ± 17 | 40 ± 8 | 38 |
| 3 | 200 | 120 | 270 ± 15 | 100 ± 12 | 43 |
| 4 | 300 | 180 | 380 ± 20 | 150 ± 10 | 54 |

TABLE 3

| | CaCl₂ | (NH₄)H₂PO₄ | |
|---|---|---|---|
| sample | (0.1M) | (0.1M) | Ca/P ratio |
| 1 | 100 | 100 | 1.1 |
| 2 | 100 | 60 | 1.5 |
| 3 | 100 | 30 | 1.6 |

A general drug carrier would naturally diffuse under the condition that it is not triggered by the external environment, and this situation is not ideal for drug systems that are required to be implanted into a human body for a long time. The drug carrier of the present invention that encapsulates the CaP and drug molecule simultaneously by means of co-deposition, may encapsulate and store a large amount of drug molecules in the shell, and it can achieve the zero-release requirement in a non-triggered state. In addition, the present process can be synthesized under room temperature and will not cause damage to the drug's efficacy.

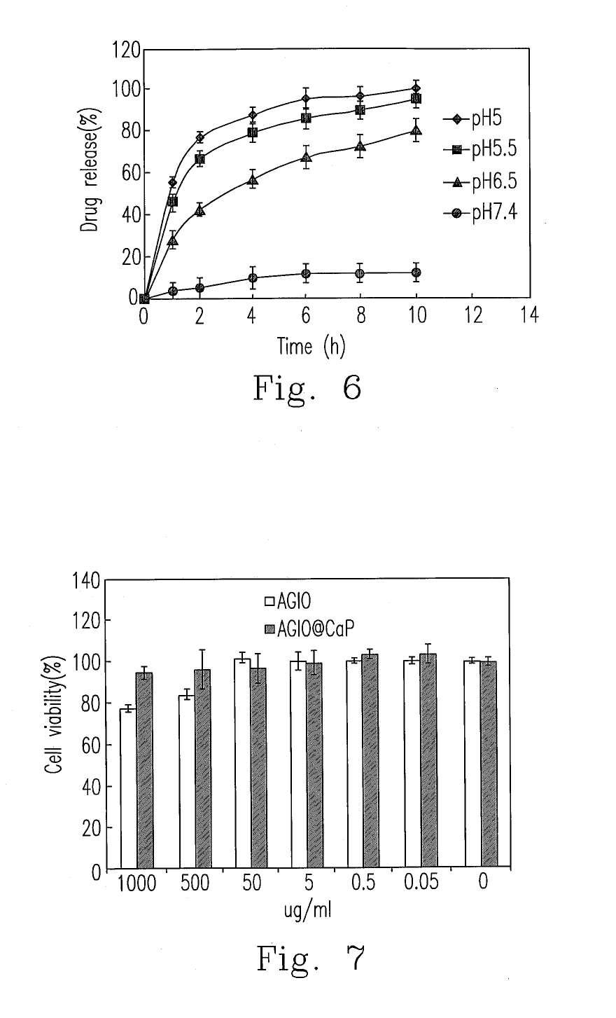

FIG. 6 is a diagram showing the drug release profiles under different acidic levels after the drug carrier of the present invention encapsulates the drug. Because DOX itself has maximum absorbance peak at 490 nm of the UV-visible spectrum, this absorbance can be utilized to calculate the released concentrations of the drug (DOX). Moreover, DOX itself has red fluorescence such that the fluorescence change of the drug carrier can be observed by a fluorescence microscope. As shown in FIG. 6, an intact CaP shell remains to coat the surface of the carrier at pH 7.4, to prevent the drug molecules from being releasing therefrom, and thereby the drug molecules can be perfectly encapsulated inside the nanocarrier. However, under an acidic environment, the absorbance representing the absorbance peak of the drug molecules obviously occurs, which represents that the drug carrier may have a different release rate under different pH values. This is because the pH value change of the external environment will cause the CaP shell of the AGIO@CaP drug carrier to be damaged by acidic dissolution, thus releasing the drug. It is clearly observed that the higher the acidic level is, the faster the CaP is disrupted, causing a higher rate of the drug to be released from the shell.

To further understand the changes in the CaP shell, TEM analysis is used to observe the damage profile of the outer CaP shell through acidic stimulus and different damage caused by different acidic levels. Under pH 5.5 for 2 hours, it is clearly observed that the thickness of the CaP shell becomes thinner, and a rough surface due to acidic etching occurs. After 8 hours, the CaP shell has completely dissolved and the AGIO is exposed. However, the extent that the CaP shell suffers from the acidic etching is more moderate at pH 6.5, the shell maintenance is more substantial and the thickness of the shell is larger, and an extensively fractured CaP shell outside the AGIO is still observed after 8 hours, and these results demonstrate the different disruption rates of the CaP under different acidic levels.

In the cellular behavior study, HeLa cells were chosen as the subject for the toxicity test of the drug carrier and ex vivo study of the drug release behavior. According to the toxicity test shown in FIG. 7, it can be known that the CaP can effectively decrease the cellular toxicity caused by the carrier due to its high biocompatibility, when the CaP coats the surface of the AGIO. By means of the tracking of the lysotracker, it can be known that the drug carrier enters the cells through the endosome. Because the endosome is acidic, the pH-sensitive drug carrier begins to dissolve the CaP, which then releases the drug molecules into the cell. The anticancer drug, DOX, carried by the drug carrier, mainly affects the cell core. The cell core is unchanged when the drug carrier reacts with the cell for 4 hours, but the drug is released due to the dissolving of the CaP when the reaction time increases to 8 hours, which enables the DOX to enter the cell core and accumulate there to where it emits red fluorescence.

The Drug Encapsulating Test for Taxol and Doxorubicin (DOX)

The amphiphilic gelatin, the hydrophobic iron oxide and hydrophobic drug (Taxol) were emulsified to form a nanosphere encapsulating the drug, co-deposition was used to enable the CaP coating on the outer layer of the sphere to form a shell, the hydrophilic drug (DOX) was encapsulated in the shell in the process of forming the CaP, and finally the pharmaceutical composition encapsulating two drugs was completed.

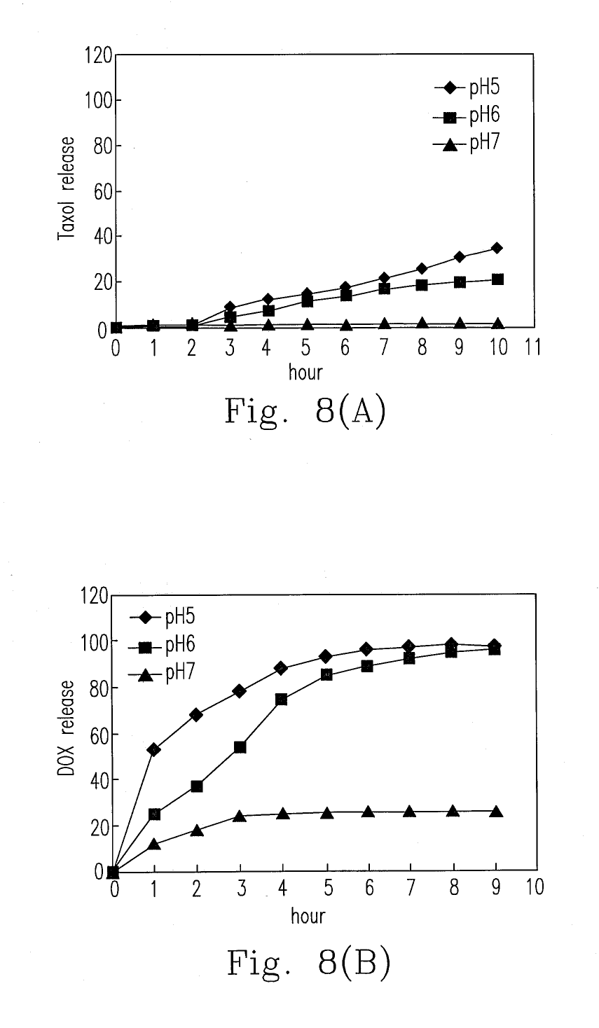

FIG. 8(A) and FIG. 8(B) show the drug release profiles under different acidic levels after the drug carrier of the present invention encapsulates two drugs, wherein FIG. 8(A) shows the release rate of the hydrophobic drug (Taxol) and FIG. 8(B) shows the release rate of the hydrophilic drug (DOX). In a neutral environment (e.g. pH 7), Taxol remains in the core and is not releases, and the release profile of DOX is about 20%. Under an environment at pH 5 and pH 6, it can be clearly observed that the release profile of Taxol increases over time after 2 hours, and the release profile of DOX reaches its peak after approximately 5 hours.

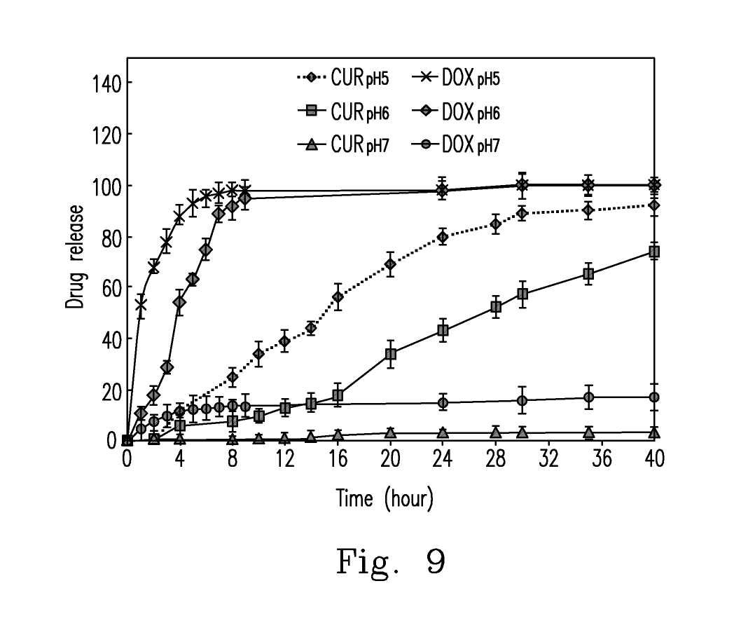

In addition, the Curcumin, a hydrophobic drug, was also successfully encapsulated in the core, and the FIG. 9 shows that the drug release profiles of the drug carrier in solutions at various pH values are similar to those in FIGS. 8(A) and 8(B). Here, it is observed that the two drugs (hydrophobic and hydrophilic) present different drug release behavior, meaning that the drug carrier of the present invention can be used to tune the drug release in different stages.

The drug carrier developed in the present invention has good pH-sensitivity and can release large amounts of drugs quickly and precisely by using the differences in the environmental pH values. Moreover, the transverse relaxivities of the drug carrier of the present invention is about 109 s⁻¹mM⁻¹ Fe, proving the MR imaging characteristics, and thus this magnetic nanocarrier is a very suitable contrast agent for MR imaging transverse relaxivities. In addition, this drug carrier is highly bio-compatible with cells. Combining these characteristics, this multi-functional nano drug carrier combines the effects of carrying multiple drugs, controlling the release and imaging, and has more applications for biomedical treatments.

### Embodiments

1. A drug carrier, including:
   a core including:
      a magnetic nanoparticle and
      an amphiphilic gelatin encapsulating the magnetic nanoparticle; and
   a calcium phosphate shell configured outside the core and used to store a hydrophilic drug.
2. A drug carrier according to Embodiment 1, wherein the amphiphilic gelatin further encapsulates a hydrophobic drug molecule, and the magnetic nanoparticle is Fe₃O₄.
3. A drug carrier according to Embodiment 1 or 2, wherein the core has a hydrophilic surface and is hydrophobic inside.
4. A drug carrier according to any one of the Embodiments 1-3, wherein the drug carrier has a size ranging from 100 nm to 400 nm, and the calcium phosphate shell has a thickness ranging from 10 nm to 150 nm and a Ca/P ratio ranging from 1.1 to 1.6.
5. A pharmaceutical composition, including:
   a nanosphere having an outer surface;
   a shell; and
   a drug mixed with the shell to be formed on the outer surface.
6. A pharmaceutical composition according to Embodiment 5, wherein the shell and the drug are deposited on the outer surface together, and the shell dissolves in an acidic environment to release the drug.
7. A pharmaceutical composition according to Embodiment 5 or 6, wherein the nanosphere is an amphiphilic gelatin such that the nanosphere has a hydrophilic surface and is hydrophobic inside.
8. A pharmaceutical composition according to any one of Embodiments 5-7, wherein the amphiphilic gelatin contains a hexanoyl.
9. A pharmaceutical composition according to any one of Embodiments 5-8, wherein the amphiphilic gelatin further encompasses at least one of a hydrophobic magnetic nanoparticle and a hydrophobic drug.
10. A pharmaceutical composition according to any one of Embodiments 5-9, wherein the hydrophobic drug is at least one selected from a group consisting of a Taxol, a Taxol derivative, a Camptothecin, a Camptothecin derivative a Curcumin and a Curcumin derivative.
11. A pharmaceutical composition according to any one of Embodiments 5-10, wherein the shell is a calcium phosphate and the drug is a hydrophilic drug.
12. A pharmaceutical composition according to any one of Embodiments 5-11, wherein the hydrophilic drug is at least one of a Doxorubicin and any derivative thereof.
14. A method for preparing a pharmaceutical composition, including steps of:
   providing a nanosphere having an outer surface;
   providing a shell material;
   providing a drug to be mixed with the shell material to form a mixture; and
   enabling the mixture to be attached to the outer surface.
14. A method according to Embodiment 13, wherein the nanosphere is an amphiphilic gelatin such that the nanoparticle has a hydrophilic surface and is hydrophobic inside.
15. A method according to Embodiment 13 or 14, wherein the amphiphilic gelatin contains a hexanoyl.
16. A method as according to any one of Embodiments 13-15, wherein the amphiphilic gelatin further encompasses at least one of a hydrophobic magnetic nanoparticle and a hydrophobic drug.
17. A method according to any one of Embodiments 13-16, wherein the hydrophobic magnetic nanoparticle is Fe₃O₄, and the hydrophobic drug is at least one selected from a group consisting of a Taxol, a Taxol derivative, a Camptothecin, a Camptothecin derivative a Curcumin and a Curcumin derivative.
18. A method according to any one of Embodiments 13-17, wherein the shell material is a calcium phosphate and the drug is a hydrophilic drug.
19. A method according to any one of Embodiments 13-18, wherein the hydrophilic drug is at least one of a Doxorubicin and any derivative thereof.
It is understood that this invention is not limited to the particular embodiments disclosed, but is intended to cover all modifications which are within the spirit and scope of the invention as defined by the appended claims, the above description, and/or shown in the attached drawings.

What is claimed is:

1. A drug carrier, comprising:
a core including:
a magnetic nanoparticle; and
an amphiphilic gelatin encapsulating the magnetic nanoparticle; and
a calcium phosphate shell configured outside the core and used to store a hydrophilic drug.

2. A drug carrier as claimed in claim 1, wherein the amphiphilic gelatin further encapsulates a hydrophobic drug molecule, and the magnetic nanoparticle is Fe₃O₄.

3. A drug carrier as claimed in claim 1, wherein the core has a hydrophilic surface and is hydrophobic inside.

4. A drug carrier as claimed in claim 1, wherein the drug carrier has a size ranging from 100 nm to 400 nm, and the calcium phosphate shell has a thickness ranging from 10 nm to 150 nm and a Ca/P ratio ranging from 1.1 to 1.6.

5. A pharmaceutical composition, comprising:
a nanosphere having an outer surface;
a shell; and
a drug mixed with the shell to be formed on the outer surface.

6. A pharmaceutical composition as claimed in claim 5, wherein the shell and the drug are deposited on the outer surface together, and the shell dissolves in an acidic environment to release the drug.

7. A pharmaceutical composition as claimed in claim 5, wherein the nanosphere is an amphiphilic gelatin such that the nanosphere has a hydrophilic surface and is hydrophobic inside.

8. A pharmaceutical composition as claimed in claim 7, wherein the amphiphilic gelatin contains a hexanoyl.

9. A pharmaceutical composition as claimed in claim 7, wherein the amphiphilic gelatin further encompasses at least one of a hydrophobic magnetic nanoparticle and a hydrophobic drug.

10. A pharmaceutical composition as claimed in claim 9, wherein the hydrophobic drug is at least one selected from a group consisting of a Taxol, a Taxol derivative, a Camptothecin, a Camptothecin derivative, a Curcumin and a Curcumin derivative.

11. A pharmaceutical composition as claimed in claim 5, wherein the shell is a calcium phosphate and the drug is a hydrophilic drug.

12. A pharmaceutical composition as claimed in claim 11, wherein the hydrophilic drug is at least one of a Doxorubicin and any derivative thereof.

13. A method for preparing a pharmaceutical composition, comprising steps of:
providing a nanosphere having an outer surface;
providing a shell material;
providing a drug to be mixed with the shell material to form a mixture; and
enabling the mixture to be attached to the outer surface.

14. A method as claimed in claim 13, wherein the nanosphere is an amphiphilic gelatin such that the nanosphere has a hydrophilic surface and is hydrophobic inside.

15. A method as claimed in claim 14, wherein the amphiphilic gelatin contains a hexanoyl.

16. A method as claimed in claim 14, wherein the amphiphilic gelatin further encompasses at least one of a hydrophobic magnetic nanoparticle and a hydrophobic drug.

17. A method as claimed in claim 16, wherein the hydrophobic magnetic nanoparticle is Fe₃O₄, and the hydrophobic drug is at least one selected from a group consisting of a Taxol, a Taxol derivative, a Camptothecin, a Camptothecin derivative, a Curcumin and a Curcumin derivative.

18. A method as claimed in claim 13, wherein the shell material is a calcium phosphate and the drug is a hydrophilic drug.

19. A method as claimed in claim 18, wherein the hydrophilic drug is at least one of a Doxorubicin and any derivative thereof.
